(19) United States
(12) Patent Application Publication
(10) Pub. No.: US 2023/0381406 A1
(43) Pub. Date: Nov. 30, 2023

(54) FILTER FOR INFUSION PUMP SYSTEMS

(71) Applicant: Tandem Diabetes Care, Inc., San Diego, CA (US)

(72) Inventors: Adam Evard, San Diego, CA (US); Michael Michaud, San Diego, CA (US)

(21) Appl. No.: 18/201,645

(22) Filed: May 24, 2023

Related U.S. Application Data
(60) Provisional application No. 63/345,273, filed on May 24, 2022.

(51) Int. Cl. A61M 5/142

(52) U.S. Cl. CPC A61M 5/14244; A61M 2205/7545

(57) ABSTRACT

Embodiments of the present disclosure relate to use of a filter in an infusion pump system. The filter can be comprised of a rigid porous plastic material that provides a three-dimensional tortuous path of small pore size that captures particulate as it flows through the filter. Filtration efficiency can be balanced with flow restriction dependent upon pore size of the filter. A range of pore sizes is achievable.

### RELATED APPLICATION

This application claims the benefit of U.S. Provisional Application No. 63/345,273 filed May 24, 2022, which is hereby fully incorporated herein by reference.

### TECHNICAL FIELD

The present disclosure relates to medical pumps for delivering medicament to a patient, and more specifically, to systems including filters for filtering medicament for delivery with medical pumps.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type I, or in some cases, type II diabetes. Some insulin injecting pumps that are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

As insulin degrades over time while in use in an insulin pump system from heat, agitation, mechanical stresses, and material interactions with the device, the insulin begins to coalesce into aggregated clumps of non-functional protein. Infusion of these protein aggregates is of clinical interest as they may induce or contribute to infusion site inflammatory and immune responses. Protein aggregate quantity and size increases over time and exposure to stressors. The typical particle size is <25 um diameter with the majority of particulate below 5 um. Prevention of the infusion of aggregated protein would be desirable.

### SUMMARY

Embodiments of the present disclosure relate to use of a filter in an infusion pump system. The filter can be comprised of a rigid porous plastic material that provides a three-dimensional tortuous path of small pore size that captures particulate as it flows through the filter. Filtration efficiency can be balanced with flow restriction dependent upon pore size of the filter. A range of pore sizes is achievable.

In embodiments, an infusion pump system can include an infusion pump configured to contain a medicament and a filter disposed in relation to the infusion pump such that a flow of insulin into or out of the infusion pump passes through the filter. The filter can include a rigid, porous plastic material that creates a three-dimensional tortuous path for the medicament flowing through the filter, the tortuous path trapping particulates in the insulin within the filter to remove the particulates from the insulin.

In embodiments, a system for delivering medicament from an infusion pump to a user can include a fluid pathway for delivering medicament from the infusion pump to an infusion site on the user and a filter disposed along the fluid pathway such that the medicament flows through the filter prior to delivery to the user, the filter comprising a rigid, porous plastic material that creates a three-dimensional tortuous path for the medicament flowing through the filter, the tortuous path trapping particulates in the insulin within the filter to remove the particulates from the insulin.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1B depict a filter for use with an infusion pump system according to an embodiment.

FIG. 2 depicts a portion of an infusion pump system including a filter according to an embodiment.

FIG. 3 depicts a portion of an infusion pump system including a filter according to an embodiment.

FIG. 4 depicts a portion of an infusion pump system including a filter according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

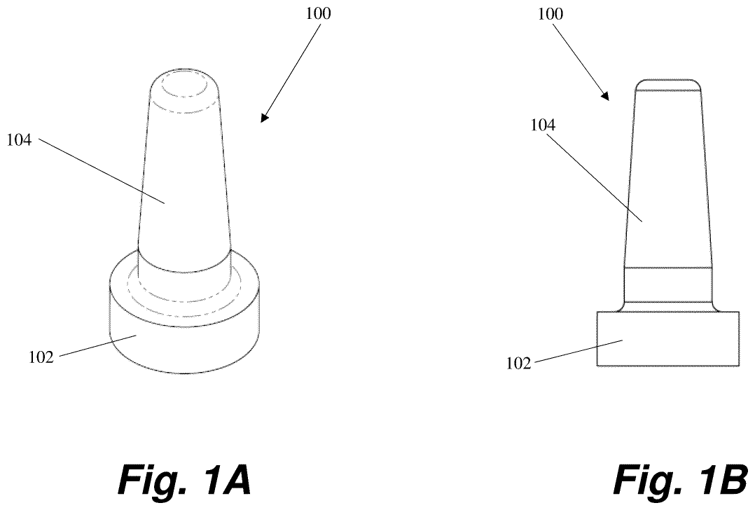

FIGS. 1A-1B depict a filter 100 for use with an infusion pump system according to an embodiment. Filter 100 can be comprised of a rigid, porous plastic material. For example, filter 100 can be comprised of polyethylene, polypropylene, hydrophilic treated PTFE (polytetrafluoroethylene) or other similar materials. Various pore sizes can be employed including, for example, pore sizes between 5 um and 250 um. In embodiments, filter 100 can include a filter head 102 and a filter body 104. The filter body 104 can be configured to be inserted into a fluid flow path in an infusion pump system with the filter head 102 serving to seat the filter 100 along the flow path. In embodiments, filter body 104 can be tapered distally of the filter head 102 to provide an interference fit with the lumen into which it is inserted. In other embodiments, filter 100 can be cylindrical, have a pass-through lumen, have more complex three-dimensional geometry to facilitate packaging and overall product design, etc. The porous filter 100 provides a three-dimensional tortuous path of small pore size that captures particulate as it flows through the filter. Porosity can be chosen to balance filtration efficiency with flow restriction.

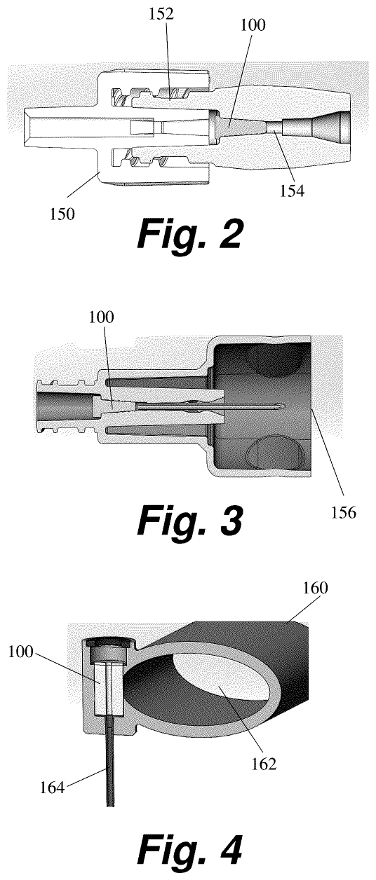

FIG. 2 depicts one embodiment of a filter 100 employed in an infusion pump system. In this configuration, the system includes a female connector 150 that may, for example, be attached to tubing extending from an infusion pump. A male connector 152 that may, for example, be connected to tubing 154 extending to an infusion set can be releasably connectable to the male connector to facilitate delivery of insulin from the pump to a user via the infusion set. The filter 100 can be press-fit into the tubing 154 adjacent the male connector 152 in order to filter the insulin flowing from the pump to the infusion set. The filter 100 can alternatively be positioned at any other location along the flow path of the insulin. Further details regarding such infusion pump systems can be found in U.S. Patent Publication No. 2011/0144586, which is incorporated by reference herein.

FIG. 3 depicts another embodiment of a filter 100 employed in an infusion pump system. In this configuration, the filter is employed with an adapter 156 used to fill a cartridge of an infusion pump system with insulin contained in an insulin storage vial. The filter 100 can be positioned within a flow path in the adapter 156 to filter the insulin as the cartridge is filled. Further details regarding such vial adapters can be found in U.S. Pat. No. 10,926,025 which is hereby incorporated by reference herein.

FIG. 4 depicts a further embodiment of a filter 100 being employed in an infusion pump system. In this embodiment, filter 100 is utilized with a patch pump 160 that delivers insulin to the user from a reservoir 162 through a cannula 164 situated directly beneath the pump 160 rather than to another location via an infusion set. The filter 100 can include a central lumen through which a soft cannula 164 extends and can be in fluid communication with the reservoir 162 to filter the insulin as it is delivered to the cannula 164. In other embodiments, the cannula 164 can be a steel cannula and the filter is positioned along the flow path between the reservoir 162 and the steel cannula without the depicted central lumen. Further details regarding such patch pumps can be found in U.S. Pat. Nos. 10,279,107 and 9,993,595, which are hereby incorporated by reference herein. A similar configuration could be employed with a pump that utilizes an infusion set, with the filter positioned at or near an outlet of the pump rather than at the junction of the pump tubing and infusion set as described with respect to FIG. 2.

A rigid, porous plastic filter as described herein provides a number of advantages over known filter elements, such as membrane filters. For example, the rigid porous filters disclosed herein are smaller and more compact than membrane filters. Such filters also provide increased filtration efficiency due to the tortuous path and better flow characteristics due to the larger pore size that can be employed compared to a membrane filter because the tortuous path can trap particulates at dead ends and corners in the path rather than through pore size alone. In addition, the ability to simply press-fit the filter along a flow path reduces the components needed for assembly and provides a fast and efficient assembly process.

Use of the filters disclosed herein to reduce delivery of particulates in insulin can contribute to extending and/or improving infusion site life and patency due to limiting infusion site inflammatory and immune responses caused by such particulates.

In embodiments, an infusion pump system can include an infusion pump configured to contain a medicament and a filter disposed in relation to the infusion pump such that a flow of insulin into or out of the infusion pump passes through the filter. The filter can include a rigid, porous plastic material that creates a three-dimensional tortuous path for the medicament flowing through the filter, the tortuous path trapping particulates in the insulin within the filter to remove the particulates from the insulin.

In some embodiments, the system further includes infusion tubing configured to connect the infusion pump to an infusion site on a body of a user to deliver medicament from the infusion pump to the infusion site and the filter is disposed within the infusion tubing.

In some embodiments, the infusion tubing includes a connector configured to connect the infusion tubing to an infusion set that extends from the connector to the infusion site and the filter is disposed in the connector.

In some embodiments, the infusion pump is configured to connect to an infusion set that extends from the infusion tubing to the infusion site and the filter is disposed in the infusion set. In some embodiments, the system further includes a cannula configured to be inserted into a body of a user at an infusion site where medicament from the infusion pump is delivered to the user.

In some embodiments, the filter is disposed around the cannula.

In some embodiments, the filter is positioned along a flow path of medicament between the infusion pump and the cannula.

In some embodiments, the system further includes an adapter configured to interface with the infusion pump to facilitate filling the infusion pump with the medicament, and the filter is disposed within the adapter.

In some embodiments, the filter comprises a filter head and a filter body.

In some embodiments, the filter body is tapered distally of the filter head such that the filter body is configured to be press fit into a lumen of a flow path of the medicament with the filter head seated proximally of the lumen.

In embodiments, a system for delivering medicament from an infusion pump to a user can include a fluid pathway for delivering medicament from the infusion pump to an infusion site on the user and a filter disposed along the fluid pathway such that the medicament flows through the filter prior to delivery to the user, the filter comprising a rigid, porous plastic material that creates a three-dimensional tortuous path for the medicament flowing through the filter, the tortuous path trapping particulates in the insulin within the filter to remove the particulates from the insulin.

In some embodiments, the fluid pathway includes infusion tubing configured to connect the infusion pump to the infusion site and the filter is disposed within the infusion tubing.

In some embodiments, the fluid pathway includes an infusion set that extends from a connector of the infusion tubing to the infusion site and the filter is disposed in the connector.

In some embodiments, fluid pathway includes an infusion set that extends from the infusion tubing to the infusion site and the filter is disposed in the infusion set.

In some embodiments, the fluid pathway includes a cannula configured to be inserted into the infusion site.

In some embodiments, the filter is disposed around the cannula.

In some embodiments, the filter is positioned along a flow path of medicament between the infusion pump and the cannula.

In some embodiments, the fluid pathway includes an adapter configured to interface with the infusion pump to facilitate filling the infusion pump with the medicament, and the filter is disposed within the adapter.

In some embodiments, the filter comprises a filter head and a filter body.

In some embodiments, the filter body is tapered distally of the filter head such that the filter body is configured to be press fit into a lumen of the fluid pathway with the filter head seated proximally of the lumen.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,279,105; 10,279,106; 10,279,107; 10,357,603; 10,357,606; 10,492,141; 10,569,016; 10,736,037; 10,888,655; 10,994,077; 11,116,901; 11,224,693; 11,291,763; 11,305,057; 11,458,246; 11,464,908; and 11,654,236 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2021/0353857; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545 and 2023/0113755 and commonly owned U.S. patent application Ser. Nos. 17/368,968; 17/896,492; 18/011,060; 18/071,814; 18/071,835; 18/075,029; 18/090,788 18/115,316; and Ser. No. 18/139,391.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An infusion pump system, comprising:
an infusion pump configured to contain a medicament; and
a filter disposed in relation to the infusion pump such that a flow of insulin into or out of the infusion pump passes through the filter, the filter comprising a rigid, porous plastic material that creates a three-dimensional tortuous path for the medicament flowing through the filter, the tortuous path trapping particulates in the insulin within the filter to remove the particulates from the insulin.

2. The infusion pump system of claim 1, further comprising infusion tubing configured to connect the infusion pump to an infusion site on a body of a user to deliver medicament from the infusion pump to the infusion site, wherein the filter is disposed within the infusion tubing.

3. The infusion pump system of claim 2, wherein the infusion tubing includes a connector configured to connect the infusion tubing to an infusion set that extends from the connector to the infusion site and wherein the filter is disposed in the connector.

4. The infusion pump system of claim 2, wherein the infusion pump is configured to connect to an infusion set that extends from the infusion tubing to the infusion site and wherein the filter is disposed in the infusion set.

5. The infusion pump system of claim 1, further comprising a cannula configured to be inserted into a body of a user at an infusion site where medicament from the infusion pump is delivered to the user.

6. The infusion pump system of claim 5, wherein the filter is disposed around the cannula.

7. The infusion pump system of claim 5, wherein the filter is positioned along a flow path of medicament between the infusion pump and the cannula.

8. The infusion pump system of claim 1, further comprising an adapter configured to interface with the infusion pump to facilitate filling the infusion pump with the medicament, and wherein the filter is disposed within the adapter.

9. The infusion pump system of claim 1, wherein the filter comprises a filter head and a filter body.

10. The infusion pump system of claim 9, wherein the filter body is tapered distally of the filter head such that the filter body is configured to be press fit into a lumen of a flow path of the medicament with the filter head seated proximally of the lumen.

11. A system for delivering medicament from an infusion pump to a user, comprising:
a fluid pathway for delivering medicament from the infusion pump to an infusion site on the user; and
a filter disposed along the fluid pathway such that the medicament flows through the filter prior to delivery to the user, the filter comprising a rigid, porous plastic material that creates a three-dimensional tortuous path for the medicament flowing through the filter, the tortuous path trapping particulates in the insulin within the filter to remove the particulates from the insulin.

12. The system of claim 11, wherein the fluid pathway includes infusion tubing configured to connect the infusion pump to the infusion site, wherein the filter is disposed within the infusion tubing.

13. The system of claim 12, wherein the fluid pathway includes an infusion set that extends from a connector of the infusion tubing to the infusion site and wherein the filter is disposed in the connector.

14. The claim 12, wherein the fluid pathway includes an infusion set that extends from the infusion tubing to the infusion site and wherein the filter is disposed in the infusion set.

15. The system of claim 1, wherein the fluid pathway includes a cannula configured to be inserted into the infusion site.

16. The system of claim 15, wherein the filter is disposed around the cannula.

17. The system of claim 15, wherein the filter is positioned along a flow path of medicament between the infusion pump and the cannula.

18. The system of claim 11, wherein the fluid pathway includes an adapter configured to interface with the infusion pump to facilitate filling the infusion pump with the medicament, and wherein the filter is disposed within the adapter.

19. The system of claim 11, wherein the filter comprises a filter head and a filter body.

20. The system of claim 19, wherein the filter body is tapered distally of the filter head such that the filter body is configured to be press fit into a lumen of the fluid pathway with the filter head seated proximally of the lumen.
